(19) United States
(12) Patent Application Publication
(10) Pub. No.: US 2024/0189123 A1
(43) Pub. Date: Jun. 13, 2024

(54) STENT WITH SELF-ADJUSTING ANTI-MIGRATION FEATURES

(71) Applicant: BOSTON SCIENTIFIC SCIMED, INC., Maple Grove, MN (US)

(72) Inventors: Martyn G. Folan, Galway (IE); Louis McNern, Donegal (IE)

(73) Assignee: BOSTON SCIENTIFIC SCIMED, INC., Maple Grove, MN (US)

(21) Appl. No.: 18/535,347

(22) Filed: Dec. 11, 2023

Related U.S. Application Data
(60) Provisional application No. 63/431,934, filed on Dec. 12, 2022.

(51) Int. Cl. A61F 2/86

(52) U.S. Cl. CPC A61F 2/86; A61F 2210/00; A61F 2220/0075

(57) ABSTRACT

A stent may include a radially expandable tubular framework includes a first end region, a second end region, a medial region positioned between the first end region and the second end region, and a lumen extending therethrough. The radially expandable tubular framework is configured to expand from a radially collapsed configuration to a radially expanded configuration. A plurality of covering strips are positioned along at least one of the first end region, the medial region, and the second end region. The plurality of covering strips fully covering the radially expandable tubular framework in the radially collapsed configuration, and the plurality of covering strips are configured to separate from one another in the radially expanded configuration to expose portions of the radially expandable tubular framework therebetween.

### CROSS REFERENCE TO RELATED APPLICATIONS

This application claims the benefit of U.S. Provisional Application No. 63/431,934, filed on Dec. 12, 2022, the disclosure of which is incorporated herein by reference.

### TECHNICAL FIELD

The present disclosure pertains to medical devices, and methods for manufacturing medical devices. More particularly, the present disclosure pertains to a stent for implantation in a body lumen, and associated methods.

### BACKGROUND

Implantable medical devices (e.g., expandable stents) may be designed to treat a variety of medical conditions in the body. For example, some expandable stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach and the colon), tracheobronchial tract, urinary tract, biliary tract, vascular system, etc.

In some instances it may be desirable to design stents to include sufficient flexibility while maintaining sufficient radial force to open the body lumen at the treatment site. However, in some stents, the compressible and flexible properties that assist in stent delivery may also result in a stent that has a tendency to migrate from its originally deployed position. For example, stents that are designed to be positioned in the esophageal or gastrointestinal tract may have a tendency to migrate due to peristalsis (i.e., the involuntary constriction and relaxation of the muscles of the esophagus, intestine, and colon which push the contents of the canal therethrough). Additionally, the generally moist and inherently lubricious environment of the esophagus, intestine, colon, etc. further contributes to a stent's tendency to migrate when deployed therein. One method to reduce stent migration may include encouraging tissue ingrowth into uncovered or bare portions of the stent. However, uncontrolled tissue ingrowth may reduce the stent patency and lead to difficulty of removal of the stent from the body lumen.

Therefore, it is desirable to provide alternative stent designs which include anti-migration features to reduce the stent's tendency to migrate. Examples of stents including anti-migration features are disclosed herein.

### BRIEF SUMMARY

This disclosure provides design, material, manufacturing method, and use alternatives for medical devices. An example medical device may include a stent.

In a first example, a stent may include a radially expandable tubular framework having a radially outward surface, a radially inward surface, a first end region, a second end region, a medial region positioned between the first end region and the second end region, and a lumen extending therethrough. The radially expandable tubular framework may be configured to expand from a radially collapsed configuration to a radially expanded configuration. A plurality of covering strips may be positioned along at least one of the first end region, the medial region, and the second end region, the plurality of covering strips may fully cover the radially expandable tubular framework in the radially collapsed configuration, and the plurality of covering strips may be configured to separate from one another in the radially expanded configuration to expose portions of the radially expandable tubular framework therebetween.

Alternatively or additionally to any of the embodiments above, each of the plurality of covering strips may extend longitudinally along the medial region of the stent, the plurality of covering strips may fully cover the medial region in the radially collapsed configuration, and the plurality of covering strips may be configured to separate from one another in the radially expanded configuration to expose portions of the medial region of the radially expandable tubular framework therebetween.

Alternatively or additionally to any of the embodiments above, a first longitudinal edge of each of the plurality of covering strips may be secured to the expandable tubular framework and a second longitudinal edge of each of the plurality of covering strips may be unsecured to the expandable tubular framework.

Alternatively or additionally to any of the embodiments above, the second longitudinal edge of each of the plurality of covering strips may be overlapped with the first longitudinal edge of an adjacent one of the plurality of covering strips in the radially collapsed configuration.

Alternatively or additionally to any of the embodiments above, the second longitudinal edges may be located radially outward of the first longitudinal edges in the radially collapsed configuration.

Alternatively or additionally to any of the embodiments above, each of the plurality of covering strips may extend circumferentially around the medial region of the stent, the plurality of covering strips may fully cover the medial region in the radially collapsed configuration, and the plurality of covering strips may be configured to separate from one another in the radially expanded configuration to expose portions of the medial region of the radially expandable tubular framework therebetween.

Alternatively or additionally to any of the embodiments above, a first circumferential edge of each of the plurality of covering strips may be secured to the expandable tubular framework and a second circumferential edge of each of the plurality of covering strips may be unsecured to the expandable tubular framework.

Alternatively or additionally to any of the embodiments above, the second circumferential edge of each of the plurality of covering strips may be overlapped with the first circumferential edge of an adjacent one of the plurality of covering strips in the radially collapsed configuration.

Alternatively or additionally to any of the embodiments above, the radially expandable tubular framework may be formed of one or more interwoven filaments defining interstices therebetween, wherein tissue may be permitted to grow into the interstices in the medial region in the radially expanded configuration and tissue may be precluded from growing into the interstices in the medial region in the radially collapsed configuration.

Alternatively or additionally to any of the embodiments above, the first end region may include a polymeric cover fully covering the interstices in the first end region, and the second end region may include a polymeric cover fully covering the interstices in the second end region.

Alternatively or additionally to any of the embodiments above, the plurality of covering strips may be separate from the polymeric cover of the first end region and the polymeric cover of the second end region.

Alternatively or additionally to any of the embodiments above, the plurality of covering strips may be positioned along the first end region, the plurality of covering strips may fully cover the first end region in the radially collapsed configuration, and the plurality of covering strips may be configured to separate from one another in the radially expanded configuration to expose portions of the first end region of the radially expandable tubular framework therebetween.

Alternatively or additionally to any of the embodiments above, one or more tie strings may be attached with the plurality of covering strips in the radially collapsed configuration, wherein the one or more tie strings may be removable from the plurality of covering strips to permit the first end region to radially expand to the radially expanded configuration.

Alternatively or additionally to any of the embodiments above, the one or more tie strings may include a tie string longitudinally interposed between adjacent ones of the plurality of covering strips.

In another example, a stent may include a radially expandable tubular framework having a radially outward surface, a radially inward surface, a first end region, a second end region, a medial region positioned between the first end region and the second end region, and a lumen extending therethrough. The radially expandable tubular framework may be configured to expand from a radially collapsed configuration to a radially expanded configuration. A first polymeric covering may fully cover the first end region, a second polymeric covering may fully cover the second end region, and a plurality of covering strips may be positioned along the medial region. The plurality of covering strips may fully cover the medial region of the radially expandable tubular framework between the first polymeric covering and the second polymeric covering in the radially collapsed configuration, and the plurality of covering strips may be configured to separate from one another in the radially expanded configuration to expose portions of the medial region of the radially expandable tubular framework therebetween.

Alternatively or additionally to any of the embodiments above, the plurality of covering strips may extend longitudinally from the first polymeric covering to the second polymeric covering.

Alternatively or additionally to any of the embodiments above, each of the plurality of covering strips may be overlapped with an adjacent one of the plurality of covering strips in the radially collapsed configuration.

In another example, a method of using a stent may include implanting a stent across a stricture in a body lumen. The stent may include a radially expandable tubular framework having a radially outward surface, a radially inward surface, a first end region, a second end region, a medial region positioned between the first end region and the second end region, and a lumen extending therethrough. A plurality of covering strips may be positioned along at least one of the first end region, the medial region, and the second end region. The method may include permitting the stent to initially expand to a first radially expanded configuration within the body lumen upon initial implantation across the stricture, wherein the plurality of covering strips may fully cover the radially expandable tubular framework in the first radially expanded configuration. The method may further include permitting the stent to further radially expand from the first radially expanded configuration to a second radially expanded configuration within the body lumen over a period of time, wherein the plurality of covering strips may be separated from one another in the second radially expanded configuration to expose portions of the radially expandable tubular framework therebetween.

Alternatively or additionally to any of the embodiments above, the plurality of covering strips may be overlapped with one another in the first radially expanded configuration, and the plurality of covering strips may be spaced apart from one another in the second radially expanded configuration.

Alternatively or additionally to any of the embodiments above, the method may include removing a tie string between adjacent one of the plurality of covering strips to permit the radially expandable tubular framework to radially expand from the first radially expanded configuration to the second radially expanded configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an illustrative stent in a radially collapsed configuration;

FIG. 1A is a cross-sectional view of the illustrative stent of FIG. 1, taken at line 1A-1A;

FIG. 2 is a side view of the illustrative stent of FIG. 1 in a radially expanded configuration;

FIG. 2A is a cross-sectional view of the illustrative stent of FIG. 2, taken at line 2A-2A;

FIG. 3 is a side view of an illustrative stent in a radially collapsed configuration;

FIG. 3A is a cross-sectional view of the illustrative stent of FIG. 3, taken at line 3A-3A;

FIG. 4 is a side view of the illustrative stent of FIG. 3 in a radially expanded configuration;

FIG. 4A is a cross-sectional view of the illustrative stent of FIG. 4, taken at line 4A-4A;

FIGS. 5 to 6 depict an illustrative method of an illustrative stent positioned across a stricture in a body lumen, moving from an initially deployed, radially expanded configuration to a post-operative, further radially expanded configuration;

FIG. 7 is a side view of an illustrative stent in a radially collapsed configuration;

FIG. 8 is a side view of the illustrative stent of FIG. 7 in a radially expanded configuration;

FIG. 9 is a side view of an illustrative stent in a radially collapsed configuration; and

FIG. 10 is a side view of the illustrative stent of FIG. 9 in a radially expanded configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver luminal patency in a patient with an esophageal stricture or other medical condition. Such stents may be used in patients experiencing dysphagia, sometimes due to esophageal cancer. An esophageal stent may allow a patient to maintain nutrition via oral intake during cancer treatment or palliation periods. Current gastrointestinal (GI) stenting regimes for the treatment of vessel strictures may rely on a self-expanding stent (SES) to resolve an underlying stricture in a body lumen while remaining in-situ. However, this type of stenting may have a high prevalence for migration, particularly in fully covered designs. Some stents may include protrusions (e.g., loops, quills, etc.) that radially protrude out from the stent body. These raised features may interact with the wall of the body lumen to reduce device migration. However, these devices may be difficult to remove or reposition as they may be difficult to purse down to lower diameters. Further, devices with these types of protrusions (or anti-migration features) may be indicated for permanent implantation. Where these devices are desired to be removable, retraction or repositioning of the devices may be limited as the angulation of the protrusions may cause vessel damage with movement of the stent. What may be desirable is an endoluminal implant or stent that includes anti-migration features to resist migration of the stent in the body lumen. In some instances, the stent can also be readily repositionable within the body lumen and/or removed from the body lumen.

While the embodiments disclosed herein are discussed with reference to esophageal stents, it is contemplated that the stents described herein may be used and sized for use in other locations such as, but not limited to: bodily tissue, bodily organs, vascular lumens, non-vascular lumens and combinations thereof, such as, but not limited to, in the coronary or peripheral vasculature, trachea, bronchi, colon, small intestine, biliary tract, urinary tract, prostate, brain, stomach and the like.

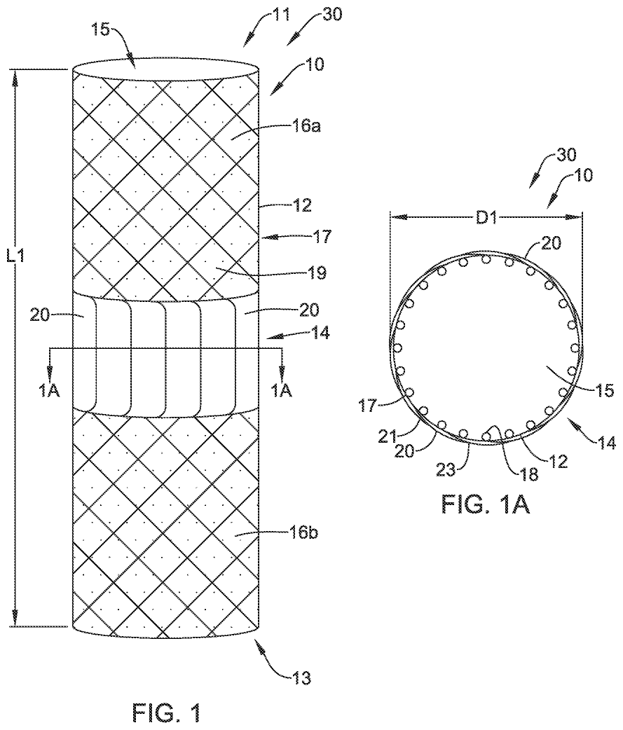

FIG. 1 is a side view of an illustrative stent 10 and FIG. 1A is a cross-sectional view of the illustrative stent 10 of FIG. 1, taken at line 1A-1A. In some cases, the stent 10 may be formed from a radially expandable tubular framework 12 (generally referred to herein as tubular framework 12), having a radially outward surface 17 and a radially inward surface 18. While the stent 10 is described as generally tubular, it is contemplated that the stent 10 may take any cross-sectional shape desired. The tubular framework 12 may further include a first end region 11, a second end region 13, and a medial region 14 positioned between the first end region 11 and the second end region 13. The tubular framework 12 may include a lumen 15 extending therethrough from the first end region 11 to the second end region 13.

It is contemplated that the tubular framework 12 can be made from a number of different materials such as, but not limited to, metals, metal alloys, shape memory alloys and/or polymers, as desired, enabling the tubular framework 12 to be expanded into shape when accurately positioned within the body. In some instances, the material may be selected to enable the tubular framework 12 to be removed with relative case as well. For example, the tubular framework 12 can be formed from alloys such as, but not limited to, Nitinol and Elgiloy®. Depending on the material selected for construction, the tubular framework 12 may be self-expanding (i.e., configured to automatically radially expand when unconstrained). In some embodiments, filaments or wires may be used to make the tubular framework 12, which in some cases may be composite filaments or wires, for example, having an outer shell made of Nitinol having a platinum core. It is further contemplated the tubular framework 12 may be formed from polymers including, but not limited to, polyethylene terephthalate (PET). In some embodiments, the tubular framework 12 may be self-expanding while in other embodiments, the tubular framework 12 may be expanded by an expansion device (such as, but not limited to a balloon inserted within the lumen 15 of the tubular framework 12). As used herein the term “self-expanding” refers to the tendency of the stent to return to a preset diameter when unrestrained from an external biasing force (for example, but not limited to, a delivery catheter or sheath). In some instances, the tubular framework 12 may include a one-way valve, such as an elastomeric slit valve or duck bill valve, positioned within the lumen 15 thereof to prevent retrograde flow of gastrointestinal fluids.

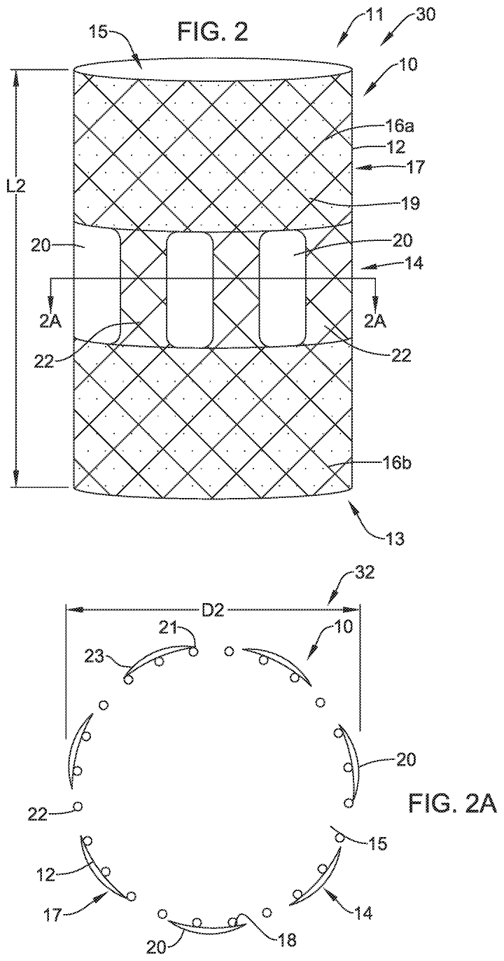

The tubular framework 12 may be configured to expand from a radially collapsed configuration 30 to a radially expanded configuration 32, as shown in FIGS. 2 to 2A. In some cases, the tubular framework 12 may be initially deployed in a body lumen to a partially expanded configuration between the radially collapsed configuration 30 and the radially expanded configuration 32. The stent 10 may be structured to extend across a stricture and to apply a radially outward pressure to the stricture in a body lumen to open the lumen and allow for the passage of foods, fluids, air, etc. When the tubular framework 12 is in the radially collapsed configuration 30, an outer diameter D₁, as shown in FIG. 1A, is reduced relative to a partially or fully radially expanded configuration (e.g., outer diameter D₂, as shown in FIG. 2A). In some cases, to reduce the diameter of the tubular framework 12 for delivery to the target location, the tubular framework 12 is elongated. For example, when the tubular framework 12 is in the radially collapsed configuration 30, the tubular framework 12 may include a first length L₁ and the first outer diameter D1. When the tubular framework 12 is deployed (moved from a delivery or radially collapsed configuration to a radially expanded configuration), the length of the tubular framework 12 decreases and the outer diameter increases. In some cases, the tubular framework 12 may experience a range of about 20% to about 40% foreshortening (e.g., the percentage by which the length of a stent decreases from its delivery configuration to its radially expanded configuration). It is contemplated that the change in length and/or the change in diameter of the tubular framework 12 may be at least partially dependent on a size (e.g., diameter) of the tubular framework 12. In some cases, a biliary stent may have a deployed diameter in the range of about 8 millimeters to about 10 millimeters, and a collapsed diameter of about 2.5 millimeters to about 3 millimeters. This may correspond to about a 60% to about 80% reduction in diameter from the radially expanded configuration 32 to the radially collapsed configuration 30. This is just an example. The reduction in diameter may be less than 60% or greater than 80%, as desired. In another example, an endoscopy stent may have a radially expanded diameter (e.g., deployed diameter) in the range of about 18 millimeters to about 23 millimeters and a radially collapsed diameter (e.g., delivery diameter) of about 6 millimeters to about 6.5 millimeters. This may correspond to about a 60% to about 80% reduction in diameter from the radially expanded configuration 32 to the radially collapsed configuration 30. This is just an example. The reduction in diameter may be less than 60% or greater than 80%, as desired.

In some cases, the tubular framework 12 may have an interwoven filament structure, fabricated from one or more interwoven filaments defining interstices 19 therebetween. In some cases, the tubular framework 12 may include a single filament knitted or otherwise interwoven with itself to form the tubular framework 12, while in other cases, the tubular framework 12 may include two or more filaments (e.g., a plurality of filaments) interwoven (e.g., wound, braided, looped, etc.) together to form the tubular framework 12. In some cases, the radially inward surface 18 and/or the radially outward surface 17 of the tubular framework 12 may be provided by the filament(s). In some cases, the radially inward surface 18 and/or the radially outward surface 17 may be entirely, substantially, or partially covered with a polymeric covering, such as a first polymeric covering 16a and/or a second polymeric covering 16b, or any other suitable type of covering or coating. The first polymeric covering 16a and the second polymeric covering 16b may be generally referred to herein as the polymeric covering 16. The polymeric covering 16 may be configured to help reduce food impaction and/or tumor or tissue ingrowth into the lumen 15 of the stent 10. In some cases, the polymeric covering 16 may be formed from any suitable material. For example, the polymeric covering 16 may be formed from silicone, polytetrafluoroethylene, polyurethane, or the like, or other materials including those disclosed herein. In some cases, the first end region 11 and the second end region 13 may include the polymeric covering 16 fully covering the interstices 19 within the first end region 11 and the second end region 13. For example, the first polymeric covering 16a may extend throughout the entire first end region 11 (both longitudinally and circumferentially) and span across the interstices in the first end region 11, and the second polymeric covering 16b may extend throughout the entire second end region 13 (both longitudinally and circumferentially) and span across the interstices in the second end region 13.

In some cases, the tubular framework 12 may include a plurality of covering strips 20 positioned along the medial region 14 of the tubular framework 12. While it is shown that the plurality of covering strips 20 are positioned along the medial region 14, it may be contemplated that the plurality of covering strips 20 may be positioned along at least one of the first end region 11, the second end region 13, and/or the medial region 14. The plurality of covering strips 20 may be separate from the polymeric covering 16 that covers the first end region 11 and the second end region 13. In some cases, the plurality of covering strips 20 may be coupled to the polymeric covering 16. In some cases, the plurality of covering strips 20 and the polymeric covering 16 may be one monolithic structure. The plurality of covering strips 20 may be configured to help reduce food impaction and/or tumor or tissue ingrowth into the lumen 15 of the stent 10. In some cases, the plurality of covering strips 20 may be formed from any suitable material. For example, the plurality of covering strips 20 may be formed from silicone, polytetrafluoroethylene, polyurethane, or the like. In some cases, coupling the plurality of covering strips 20 to the tubular framework 12 may include adhesive bonding, thermal bonding, molding, coating, dip coating, extruding, or the like. These are just examples.

In some cases, each of the plurality of covering strips 20 may extend longitudinally from the first polymeric covering 16a to the second polymeric covering 16b along the medial region 14 of the tubular framework 12. The plurality of covering strips 20 may fully cover the medial region 14 of the tubular framework 12 in the radially collapsed configuration 30, as shown in FIGS. 1 to 1A. The plurality of covering strips 20 may each include a first longitudinal edge 21 secured to the tubular framework 12 and a second longitudinal edge 23 unsecured to the tubular framework 12. In some cases, when the tubular framework 12 is in the radially collapsed configuration 30, the second longitudinal edges 23 may be located radially outward of the first longitudinal edges 21. In such cases, the second longitudinal edge 23 of each of the plurality of covering strips 20 may be located radially outward of and overlapped with the first longitudinal edge 21 of an adjacent one of the plurality of covering strips 20, thereby providing a full covering over the medial region 14 when the tubular framework 12 is in the radially collapsed configuration 30. Thus, when the tubular framework 12 is in the radially collapsed configuration 30, tissue may be precluded from growing into the interstices 19 of the medial region 14 of the tubular framework 12 and into the lumen 15.

FIG. 2 is a side view of the illustrative stent 10 of FIG. 1 in the radially expanded configuration 32, and FIG. 2A is a cross-sectional view of the illustrative stent 10 of FIG. 2, taken at line 2A-2A. As discussed with reference to FIGS. 1 to 1A, when the tubular framework 12 is initially deployed (moved from a delivery or radially collapsed configuration to an expanded configuration during a medical procedure), the length of the tubular framework 12 may decrease and the outer diameter may increase. In some cases, the tubular framework 12 may be placed across a stricture in a body lumen in the initially deployed, radially expanded configuration. The tubular framework 12 may move from the initially deployed, radially expanded configuration to the radially expanded configuration 32 shown in FIG. 2 as the stricture resolves, such that a diameter of the lumen through the stricture increases. In such cases, the tubular framework 12 may expand to a greater outer diameter as the stricture resolves. For example, the tubular framework 12 may expand to a second, shorter length L₂ and a second, increased outer diameter D₂.

As discussed with reference to FIGS. 1 to 1A, the tubular framework 12 may include the plurality of covering strips 20. When the tubular framework 12 is in the radially expanded configuration 32, the plurality of covering strips 20 may be configured to circumferentially separate from one another to expose portions 22 of the tubular framework 12 (i.e., the filament(s)) therebetween. The exposed portions 22 (e.g., bare or uncovered) of the tubular framework 12 may be free from or devoid of the polymeric covering 16, and may expose the interstices 19 of the medial region 14 of the tubular framework 12 opening into the lumen 15. Tissue may be permitted to grow into the interstices 19 in the medial region 14 when the tubular framework 12 is in the radially expanded configuration 32, thereby reducing migration of the stent 10.

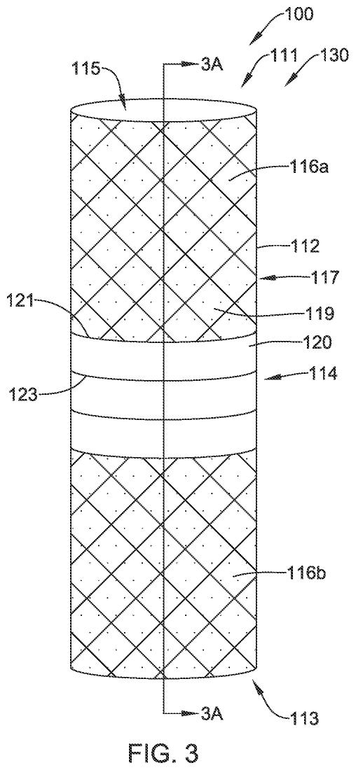

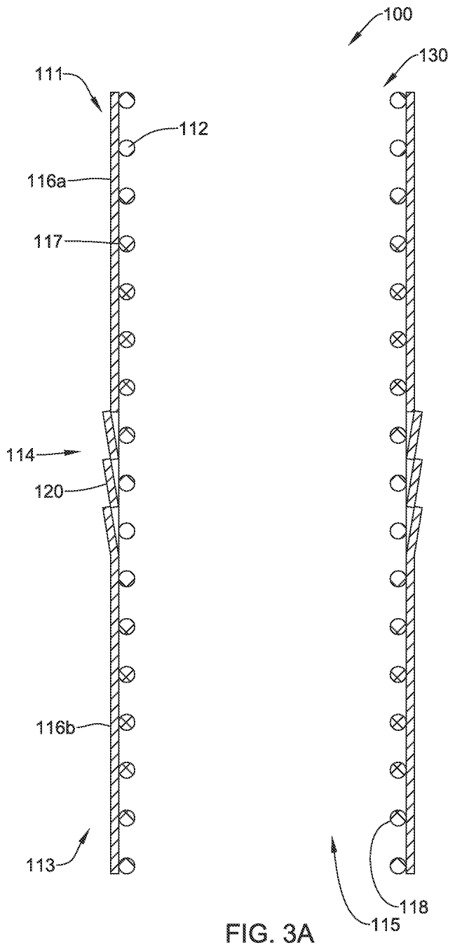

FIG. 3 is a side view of an illustrative stent 100 in a radially collapsed configuration, and FIG. 3A is a cross-sectional view of the illustrative stent 100 of FIG. 3, taken at line 3A-3A. In some cases, the stent 100 may be formed from a radially expandable tubular framework 112 (generally referred to herein as tubular framework 112), having a radially outward surface 117 and a radially inward surface 118. While the stent 100 is described as generally tubular, it is contemplated that the stent 100 may take any cross-sectional shape desired. The tubular framework 112 may further include a first end region 111, a second end region 113, and a medial region 114 positioned between the first end region 111 and the second end region 113. The tubular framework 112 may include a lumen 115 extending therethrough from the first end region 111 to the second end region 113.

It is contemplated that the tubular framework 112 can be made from a number of different materials such as, but not limited to, metals, metal alloys, shape memory alloys and/or polymers, as desired, enabling the tubular framework 112 to be expanded into shape when accurately positioned within the body. In some instances, the material may be selected to enable the tubular framework 112 to be removed with relative case as well. For example, the tubular framework 112 can be formed from alloys such as, but not limited to, Nitinol and Elgiloy®. Depending on the material selected for construction, the tubular framework 112 may be self-expanding (i.e., configured to automatically radially expand when unconstrained). In some embodiments, filaments or wires may be used to make the tubular framework 112, which in some cases may be composite filaments or wires, for example, having an outer shell made of Nitinol having a platinum core. It is further contemplated the tubular framework 112 may be formed from polymers including, but not limited to, polyethylene terephthalate (PET). In some embodiments, the tubular framework 112 may be self-expanding while in other embodiments, the tubular framework 112 may be expanded by an expansion device (such as, but not limited to a balloon inserted within the lumen 115 of the tubular framework 112). As used herein the term “self-expanding” refers to the tendency of the stent to return to a preset diameter when unrestrained from an external biasing force (for example, but not limited to, a delivery catheter or sheath). In some instances, the tubular framework 112 may include a one-way valve, such as an elastomeric slit valve or duck bill valve, positioned within the lumen 115 thereof to prevent retrograde flow of gastrointestinal fluids.

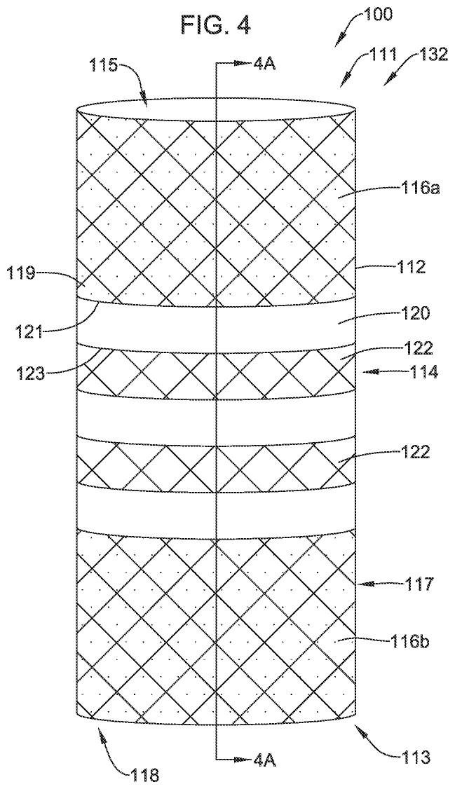

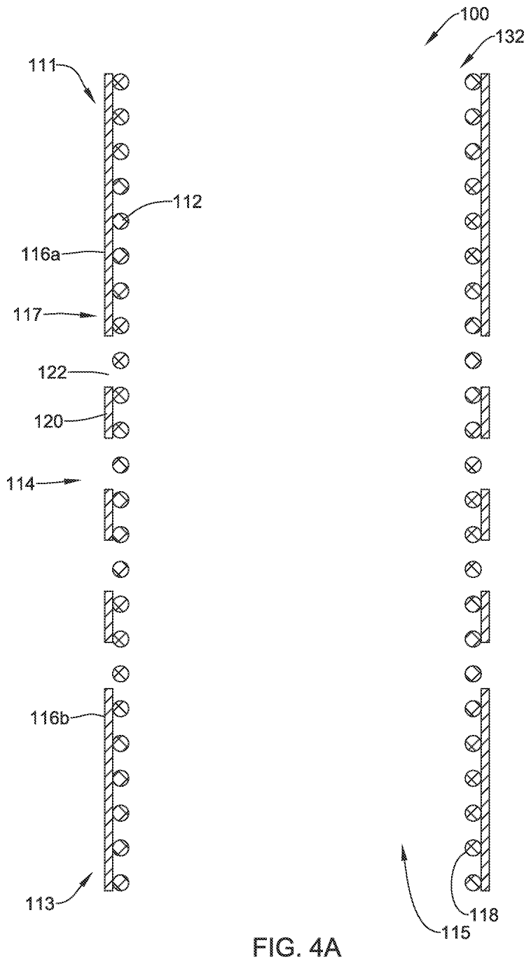

The tubular framework 112 may be configured to expand from a radially collapsed configuration 130 to a radially expanded configuration 132, as shown in FIGS. 4 to 4A. In some cases, the tubular framework 112 may be initially deployed in a body lumen to a partially expanded configuration between the radially collapsed configuration 30 and the radially expanded configuration 132. The stent 100 may be structured to extend across a stricture and to apply a radially outward pressure to the stricture in a body lumen to open the lumen and allow for the passage of foods, fluids, air, etc. When the tubular framework 112 is in the radially collapsed configuration 130, an outer diameter is reduced relative to a partially or fully radially expanded configuration. In some cases, to reduce the diameter of the tubular framework 112 for delivery to the target location, the tubular framework 112 is elongated. For example, when the tubular framework 112 is in the radially collapsed configuration 130, the tubular framework 112 may include a first length and the first outer diameter. When the tubular framework 112 is deployed (moved from a delivery or radially collapsed configuration to a radially expanded configuration), the length of the tubular framework 112 decreases and the outer diameter increases. In some cases, the tubular framework 112 may experience a range of about 20% to about 40% foreshortening (e.g., the percentage by which the length of a stent decreases from its delivery configuration to its radially expanded configuration). It is contemplated that the change in length and/or the change in diameter of the tubular framework 112 may be at least partially dependent on a size (e.g., diameter) of the tubular framework 112. In some cases, a biliary stent may have a deployed diameter in the range of about 6 millimeters to about 10 millimeters, and a collapsed diameter of about 2.5 millimeters to about 3 millimeters. This may correspond to about a 60% to about 80% reduction in diameter from the radially expanded configuration 132 to the radially collapsed configuration 130 (e.g., a delivery configuration). This is just an example. The reduction in diameter may be less than 60% or greater than 80%, as desired. In another example, an endoscopy stent may have a radially expanded diameter (e.g., deployed diameter) in the range of about 18 millimeters to about 23 millimeters and a radially collapsed diameter (e.g., delivery diameter) of about 6 millimeters to about 6.5 millimeters. This may correspond to about a 60% to about 80% reduction in diameter from the radially expanded configuration 132 to the radially collapsed configuration 130. This is just an example. The reduction in diameter may be less than 60% or greater than 80%, as desired.

In some cases, the tubular framework 112 may have an interwoven filament structure, fabricated from one or more interwoven filaments defining interstices 119 therebetween. In some cases, the tubular framework 112 may include a single filament knitted or otherwise interwoven with itself to form the tubular framework 112, while in other cases, the tubular framework 112 may include two or more filaments (e.g., a plurality of filaments) interwoven (e.g., wound, braided, looped, etc.) together to form the tubular framework 112. In some cases, the radially inward surface 118 and/or the radially outward surface 117 of the tubular framework 112 may be provided by the filament(s). In some cases, the radially inward surface 118 and/or the radially outward surface 117 may be entirely, substantially, or partially covered with a polymeric covering, such as a first polymeric covering 116a and/or a second polymeric covering 116b, or any other suitable type of covering or coating. The first polymeric covering 116a and the second polymeric covering 116b may be generally referred to herein as the polymeric covering 116. The polymeric covering 116 may be configured to help reduce food impaction and/or tumor or tissue ingrowth into the lumen 115 of the stent 100. In some cases, the polymeric covering 116 may be formed from any suitable material. For example, the polymeric covering 116 may be formed from silicone, polytetrafluoroethylene, polyurethane, or the like, or other materials including those disclosed herein. In some cases, the first end region 111 and the second end region 113 may include the polymeric covering 116 fully covering the interstices 119 within the first end region 111 and the second end region 113. For example, the first polymeric covering 116a may extend throughout the entire first end region 111 (both longitudinally and circumferentially) and span across the interstices in the first end region 111, and the second polymeric covering 116b may extend throughout the entire second end region 113 (both longitudinally and circumferentially) and span across the interstices in the second end region 113.

In some cases, the tubular framework 112 may include a plurality of covering strips 120 positioned along the medial region 114 of the tubular framework 112. While it is shown that the plurality of covering strips 120 are positioned along the medial region 114, it may be contemplated that the plurality of covering strips 120 may be positioned along at least one of the first end region 111, the second end region 113, and/or the medial region 114. The plurality of covering strips 120 may be separate from the polymeric covering 116 that covers the first end region 111 and the second end region 113. In some cases, the plurality of covering strips 120 may be coupled to the polymeric covering 116. In some cases, the plurality of covering strips 120 and the polymeric covering 116 may be one monolithic structure. The plurality of covering strips 120 may be configured to help reduce food impaction and/or tumor or tissue ingrowth into the lumen 115 of the stent 100. In some cases, the plurality of covering strips 120 may be formed from any suitable material. For example, the plurality of covering strips 120 may be formed from silicone, polytetrafluoroethylene, polyurethane, or the like. In some cases, coupling the plurality of covering strips 120 to the tubular framework 112 may include adhesive bonding, thermal bonding, molding, coating, dip coating, extruding, or the like. These are just examples.

In some cases, each of the plurality of covering strips 120 may extend circumferentially around the medial region 114 of the tubular framework 112 between the first polymeric covering 116a and the second polymeric covering 116b. The plurality of covering strips 120 may fully cover the medial region 114 of the tubular framework 112 in the radially collapsed configuration 130, as shown in FIGS. 3 to 3A. The plurality of covering strips 120 may each include a first circumferential edge 121 secured to the tubular framework 112 and a second circumferential edge 123 unsecured to the tubular framework 112. In some cases, when the tubular framework 112 is in the radially collapsed configuration 130, the second circumferential edges 123 may be located radially outward of the first circumferential edges 121. In such cases, the second circumferential edge 123 of each of the plurality of covering strips 120 may be located radially outward of and overlapped with the first circumferential edge 121 of an adjacent one of the plurality of covering strips 120, thereby providing a full covering over the medial region 114 when the tubular framework 112 is in the radially collapsed configuration 130. Thus, when the tubular framework 112 is in the radially collapsed configuration 130, tissue may be precluded from growing into the interstices 119 of the medial region 114 of the tubular framework 112 and into the lumen 115.

FIG. 4 is a side view of the illustrative stent 100 of FIG. 3 in a radially expanded configuration 132, and FIG. 4A is a cross-sectional view of the illustrative stent 100 of FIG. 4, taken at line 4A-4A. As discussed with reference to FIGS. 3 to 3A, when the tubular framework 112 is initially deployed (moved from a delivery or radially collapsed configuration to an expanded configuration during a medical procedure), the length of the tubular framework 112 may decrease and the outer diameter may increase. In some cases, the tubular framework 112 may be placed across a stricture in a body lumen in the initially deployed, radially expanded configuration. The tubular framework 112 may move from the initially deployed, radially expanded configuration to the radially expanded configuration 132 shown in FIG. 4 as the stricture resolves, such that a diameter of the lumen through the stricture increases. In such cases, the tubular framework 112 may expand to a greater outer diameter as the stricture resolves. For example, the tubular framework 112 may expand to a second, shorter length and a second, increased outer diameter.

As discussed with reference to FIGS. 3 to 3A, the tubular framework 112 may include the plurality of covering strips 120. When the tubular framework 112 is in the radially expanded configuration 132, the plurality of covering strips 120 may be configured to longitudinally separate from one another to expose portions 122 of the tubular framework 112 (i.e., the filament(s)) therebetween. The exposed portions 122 (e.g., bare or uncovered) of the tubular framework 112 may be free from or devoid of the polymeric covering 116, and may expose the interstices 119 of the medial region 114 of the tubular framework 112 opening into the lumen 115. Tissue may be permitted to grow into the interstices 119 in the medial region 14 when the tubular framework 112 is in the radially expanded configuration 132, thereby reducing migration of the stent 100.

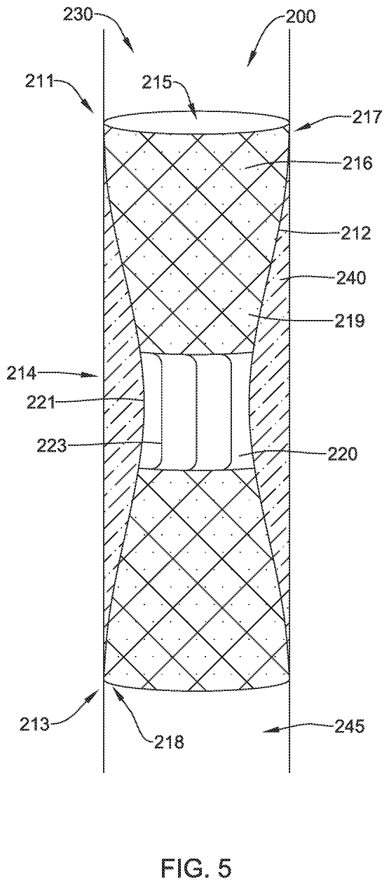

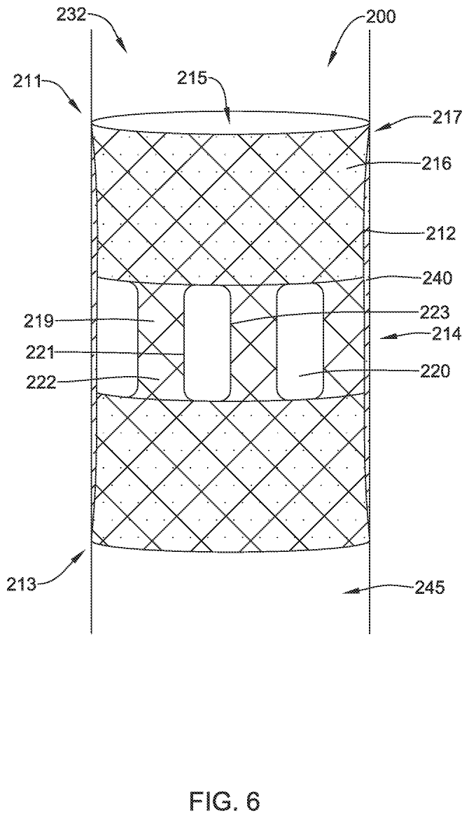

FIGS. 5 to 6 depict an illustrative method of using an illustrative stent 200, wherein the stent 200 is initially positioned across a stricture 240 in a body lumen and expanded to an initial radially expanded configuration 230, shown in FIG. 5, during a medical procedure, and subsequently further radially expanded post-implantation in the body lumen to a second, further radially expanded configuration 232, shown in FIG. 6, at a subsequent time after the medical procedure (e.g., several days or more, or several weeks or more after the medical procedure). The stent 200 may be considered to be an example of the stent 10, 100, 300, 400, discussed herein. The stent 200 may be formed from a radially expandable tubular framework 212 (generally referred to herein as tubular framework 212), having a radially outward surface 217 and a radially inward surface 218. While the stent 200 is described as generally tubular, it is contemplated that the stent 200 may take any cross-sectional shape desired. The tubular framework 212 may further include a first end region 211, a second end region 213, and a medial region 214 positioned between the first end region 211 and the second end region 213. The tubular framework 212 may include a lumen 215 extending therethrough from the first end region 211 to the second end region 213.

The tubular framework 212 may include a plurality of covering strips 220 positioned along the medial region 214 of the tubular framework 212. While it is shown that the plurality of covering strips 220 are positioned along the medial region 214, it may be contemplated that the plurality of covering strips 220 may be positioned along at least one of the first end region 211, the second end region 213, and/or the medial region 214. The plurality of covering strips 220 may be configured to help reduce food impaction and/or tumor or tissue ingrowth into the lumen 215 of the stent 200. In some cases, the plurality of covering strips 220 may be formed from any suitable material. For example, the plurality of covering strips 220 may be formed from silicone, polytetrafluoroethylene, polyurethane, or the like. In some cases, coupling the plurality of covering strips 220 to the tubular framework 212 may include adhesive bonding, thermal bonding, molding, coating, dip coating, extruding, or the like. These are just examples.

In some cases, the plurality of covering strips 220 may fully cover the medial region 214 of the stent 200 in an initially deployed, first radially expanded configuration 230, as shown in FIG. 5. In some cases, the plurality of covering strips 220 may fully cover the first end region 211 of the stent 200 in the initially deployed, first radially expanded configuration 230, and/or, the plurality of covering strips 220 may fully cover the second end region 213 of the stent 200, although not explicitly shown. The plurality of covering strips 220 may each include a first edge 221 secured to the tubular framework 212 and a second edge 223 unsecured to the tubular framework 212. In some cases, when the stent 200 is in the first radially expanded configuration 230, the second edges 223 may be located radially outward of the first edges 221. In such cases, the second edge 223 of each of the plurality of covering strips 220 may be radially outward of and overlapped with the first edge 221 of an adjacent one of the plurality of covering strips 220, thereby providing a full covering over the medial region 214, the first end region 211, or the second end region 213 when the stent 200 is in the initially deployed, first radially expanded configuration 230. Thus, when the stent 200 is in the first radially expanded configuration 230, tissue may be precluded from growing into the interstices 219 of the medial region 214 (or the first end region 211/second end region 213) of the stent 200.

In some cases, the plurality of covering strips 220 may be positioned along the first end region 211, and the plurality of covering strips 220 may fully cover the first end region 211 while the stent 200 is in the first radially expanded configuration. In some cases, the plurality of covering strips 220 may include one or more tie strings attached with the plurality of covering strips 220. In some cases, the one or more tie strings may include a tie string longitudinally interposed between adjacent ones of the plurality of covering strips 220. The tie strings may retain the plurality of covering strips 220 in an abutting and/or overlapping manner and be configured to be withdrawn from the plurality of covering strips 220 to permit the covering strips 220 separate from adjacent covering strips 220 and move apart from adjacent covering strips 220.

In some cases, when the stent 200 is in the second, further radially expanded configuration 232, such as after being implanted across the stricture 240 for a period of time, the plurality of covering strips 220 may be configured to be spaced apart, e.g., separated from one another, to expose portions 222 of the tubular framework 212 therebetween. The exposed portions 222 (e.g., bare or uncovered) of the tubular framework 212 may be free from or devoid of a polymeric covering 216, and may expose the interstices 219 of the medial region 214 of the tubular framework 212 opening into the lumen 215. Tissue may be permitted to grow into the interstices 219 in the medial region 214 when the tubular framework 212 is in the second, further radially expanded configuration 232, thereby reducing migration of the stent 200.

In some cases, the method may include implanting the stent 200 across the stricture 240 in a body lumen 245 during a medical procedure. For example, in some cases, the body lumen 245 may include, but not limited to: bodily tissue, bodily organs, vascular lumens, non-vascular lumens and combinations thereof, such as, but not limited to, in the coronary or peripheral vasculature, esophagus, trachea, bronchi, colon, small intestine, biliary tract, urinary tract, prostate, brain, stomach and the like. The stent 200 may be structured to extend across the stricture 240 and to apply a radially outward pressure to the stricture 240 in the body lumen 245 to open the body lumen 245 and allow for the passage of foods, fluids, air, etc. The method may include permitting the stent 200 to initially expand to the first radially expanded configuration 230 within the body lumen 245 upon initial implantation across the stricture, where the plurality of covering strips 220 fully cover the tubular framework 212 throughout the medial region 214 of the tubular framework 212. In other words, when the stent 200 is initially implanted across the stricture 240 in the body lumen 245, the tubular framework 212 may be fully covered (i.e., all of the interstices of the tubular framework 212 are covered with a covering, either the covering 216 on the first end region 211 and the second end region 213, or the covering strips 220 on the medial region 214), such that tissue is prevented from growing into the interstices of the tubular framework 212 into the lumen 215. The method may include permitting the stent 200 to further radially expand from the first radially expanded configuration 230 to a second radially expanded configuration 232 within the body lumen 245 over a period of time subsequent to implantation of the stent 200 (e.g., over a period of days and/or weeks after the surgical procedure in which the stent 200 was implanted) as the stricture resolves, wherein the plurality of covering strips 220 are separated from one another in the second, further radially expanded configuration 232 to expose portions 222 of the tubular framework 212 therebetween, as shown in FIG. 6. The exposed portions 222 may include uncovered or open interstices 219 between filaments of the tubular framework 212 permitting tissue ingrowth therethrough. Thus, portions of the medial region 214 of the tubular framework 212 may transition from being covered by the covering strips 220 in the first, initially deployed radially expanded configuration at the time of implantation in a body lumen, shown in FIG. 5, to being uncovered to defined the exposed portions 222 of the tubular framework 212 in the further radially expanded configuration, shown in FIG. 6 at a subsequent post-operative time (e.g., several days and/or weeks after implantation of the stent 200).

It is noted that at the point of initial implantation with the medial region 214 positioned at the stricture 240, the stricture 240 may prevent or inhibit full radial expansion of the medial region 214 such that the initial expanded diameter of the medial region 214 of the stent 200 may be less than the initial expanded diameter of the first end region 211 and/or the initial expanded diameter the second end region 213. The enlarged first end region 211 and/or the enlarged second end region 213 relative to the medial region 214 may prevent migration of the stent 200 in the body lumen 245 upon initial implantation of the stent 200. However, over time, as the stricture 240 resolves the medial region 214 may continue to radially expand to a further radially expanded configuration, shown in FIG. 6, having an outer diameter greater than the outer diameter of the initially radially expanded configuration of the medial region 214, shown in FIG. 5. In instances in which the stent 200 remains fully covered once the medial region 214 may further expanded, the stent 200 may be prone to migrate in the body lumen. However, as shown in FIG. 6, uncovering or exposing the exposed portions 222 of the tubular framework 212 permits tissue ingrowth into the interstices 219 of the exposed portions 222, providing anti-migration capabilities to the stent 200.

In some cases, the method may include removing one of the plurality of tie strings between adjacent ones of the plurality of covering strips 220 to permit the tubular framework 212 to further radially expand from the first, initially radially expanded configuration 230 to the second, further radially expanded configuration 232. Other removable structures that may be selectively removed from the covering strips 220 to permit the tubular framework 212 to expand to expose some portions of the tubular framework 212 between adjacent covering strips 220 are also contemplated.

In some cases, when the stent 200 moves from the first, initially deployed radially expanded configuration 230 to the second, post-operative further radially expanded configuration 232, the length of the tubular framework 212 may decrease and the outer diameter may increase. The tubular framework 212 may move from the first radially expanded configuration 230 to the second radially expanded configuration 232 as the stricture 240 resolves, such that a diameter of the lumen through the stricture 240 increases, as shown in FIG. 6. In such cases, the tubular framework 212 (e.g., the medial region 214) may expand over time to a larger outer diameter as the stricture 240 resolves. For example, the medial region 214 of the tubular framework 212 may expand to a second, larger outer diameter from a first, initially deployed outer diameter after a period of time (e.g., several days and/or weeks) after initial implantation.

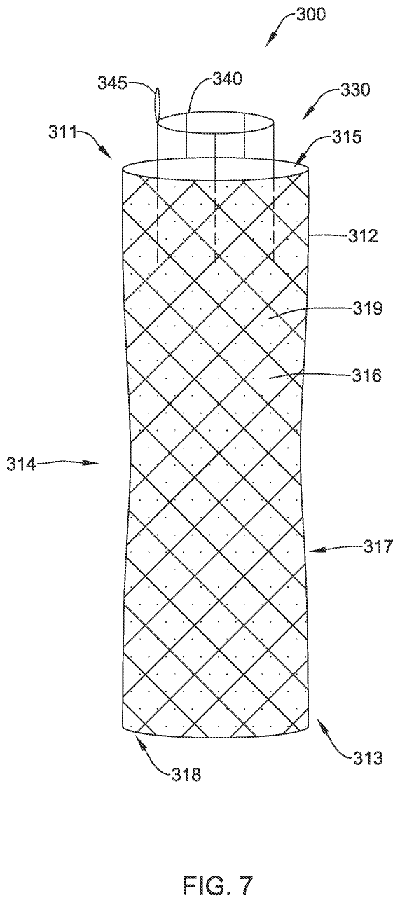

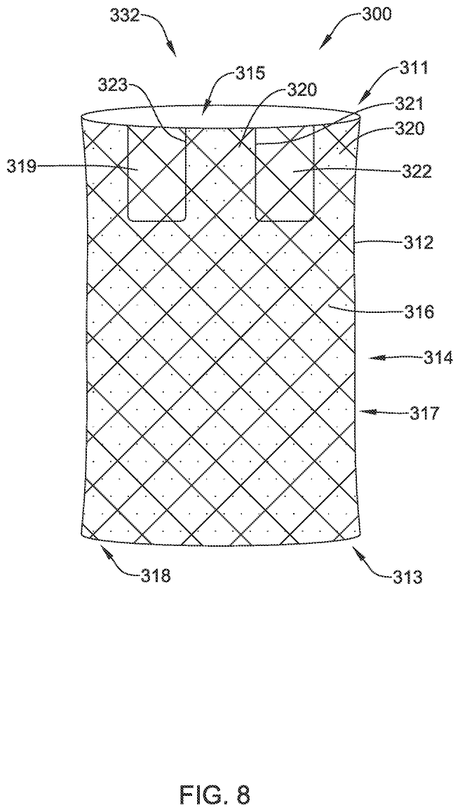

FIG. 7 is a side view of an illustrative stent 300 in a radially collapsed configuration, and FIG. 8 is a side view of the illustrative stent 300 of FIG. 7 in a radially expanded configuration 332. In some cases, the stent 300 may be formed from a radially expandable tubular framework 312 (generally referred to herein as tubular framework 312), having a radially outward surface 317 and a radially inward surface 318. While the stent 300 is described as generally tubular, it is contemplated that the stent 300 may take any cross-sectional shape desired. The tubular framework 312 may further include a first end region 311, a second end region 313, and a medial region 314 positioned between the first end region 311 and the second end region 313. The tubular framework 312 may include a lumen 315 extending therethrough from the first end region 311 to the second end region 313.

It is contemplated that the tubular framework 312 can be made from a number of different materials such as, but not limited to, metals, metal alloys, shape memory alloys and/or polymers, as desired, enabling the tubular framework 312 to be expanded into shape when accurately positioned within the body. In some instances, the material may be selected to enable the tubular framework 312 to be removed with relative ease as well. For example, the tubular framework 312 can be formed from alloys such as, but not limited to, Nitinol and Elgiloy®. Depending on the material selected for construction, the tubular framework 312 may be self-expanding (i.e., configured to automatically radially expand when unconstrained). In some embodiments, filaments or wires may be used to make the tubular framework 312, which in some cases may be composite filaments or wires, for example, having an outer shell made of Nitinol having a platinum core. It is further contemplated the tubular framework 312 may be formed from polymers including, but not limited to, polyethylene terephthalate (PET). In some embodiments, the tubular framework 312 may be self-expanding while in other embodiments, the tubular framework 312 may be expanded by an expansion device (such as, but not limited to a balloon inserted within the lumen 315 of the tubular framework 312). As used herein the term “self-expanding” refers to the tendency of the stent to return to a preset diameter when unrestrained from an external biasing force (for example, but not limited to, a delivery catheter or sheath). In some instances, the tubular framework 312 may include a one-way valve, such as an elastomeric slit valve or duck bill valve, positioned within the lumen 315 thereof to prevent retrograde flow of gastrointestinal fluids.

The tubular framework 312 may be configured to expand from a radially collapsed configuration 330, shown in FIG. 7, to a radially expanded configuration 332, shown in FIG. 8. In some cases, the tubular framework 312 may be initially deployed in a body lumen to a partially expanded configuration between the radially collapsed configuration 330 and the radially expanded configuration 332. The stent 300 may be structured to extend across a stricture and to apply a radially outward pressure to the stricture in a body lumen to open the lumen and allow for the passage of foods, fluids, air, etc. When the tubular framework 312 is in the radially collapsed configuration 330, an outer diameter, is reduced relative to a partially or fully radially expanded configuration. In some cases, to reduce the diameter of the tubular framework 312 for delivery to the target location, the tubular framework 12 is elongated. For example, when the tubular framework 12 is in the radially collapsed configuration 30, the tubular framework 12 may include a first length L₁ and the first outer diameter D1. When the tubular framework 12 is deployed (moved from a delivery or radially collapsed configuration to a radially expanded configuration), the length of the tubular framework 312 decreases and the outer diameter increases. In some cases, the tubular framework 312 may experience a range of about 20% to about 40% foreshortening (e.g., the percentage by which the length of a stent decreases from its delivery configuration to its radially expanded configuration). It is contemplated that the change in length and/or the change in diameter of the tubular framework 312 may be at least partially dependent on a size (e.g., diameter) of the tubular framework 312. In some cases, a biliary stent may have a deployed diameter in the range of about 8 millimeters to about 10 millimeters, and a collapsed diameter of about 2.5 millimeters to about 3 millimeters. This may correspond to about a 60% to about 80% reduction in diameter from the radially expanded configuration 332 to the radially collapsed configuration 330. This is just an example. The reduction in diameter may be less than 60% or greater than 80%, as desired. In another example, an endoscopy stent may have a radially expanded diameter (e.g., deployed diameter) in the range of about 18 millimeters to about 23 millimeters and a radially collapsed diameter (e.g., delivery diameter) of about 6 millimeters to about 6.5 millimeters. This may correspond to about a 60% to about 80% reduction in diameter from the radially expanded configuration 332 to the radially collapsed configuration 330. This is just an example. The reduction in diameter may be less than 60% or greater than 80%, as desired.

In some cases, the tubular framework 312 may have an interwoven filament structure, fabricated from one or more interwoven filaments defining interstices 319 therebetween. In some cases, the tubular framework 312 may include a single filament knitted or otherwise interwoven with itself to form the tubular framework 312, while in other cases, the tubular framework 312 may include two or more filaments (e.g., a plurality of filaments) interwoven (e.g., wound, braided, looped, etc.) together to form the tubular framework 312. In some cases, the radially inward surface 318 and/or the radially outward surface 317 of the tubular framework 312 may be provided by the filament(s). In some cases, the radially inward surface 318 and/or the radially outward surface 317 may be entirely, substantially, or partially covered with a polymeric covering 316, or any other suitable type of covering or coating. The polymeric covering 316 may be configured to help reduce food impaction and/or tumor or tissue ingrowth into the lumen 315 of the stent 300. In some cases, the polymeric covering 316 may be formed from any suitable material. For example, the polymeric covering 316 may be formed from silicone, polytetrafluoroethylene, polyurethane, or the like, or other materials including those disclosed herein. In some cases, as shown in FIGS. 7 to 8, the first end region 311, the second end region 313, and the medial region 314 may include the polymeric covering 316 and span across the interstices 319 in the first end region 111, the second end region 313, and the medial region 314.

The stent 300 may include one or more tie strings 340 attached to and extending along a portion of the polymeric covering 316, such as along the first end region 311, in the radially collapsed configuration 330. The one or more tie strings 340 may include a retrieval tether 345, or suture secured or otherwise provided with the one or more tie strings 340. As discussed herein, the tie strings 340 may be selectively removed from the polymeric covering 316, such as by medical personnel grasping the retrieval tether 345 and withdrawing the retrieval tether 345 and attached tie strings 340 from the polymeric covering 316. Removing the tie strings 340 may permit portions of the polymeric covering 316 (e.g., covering strips 320) to move away from one another to expose portions of the tubular framework 312 therebetween, as shown in FIG. 8.

In some cases, the tubular framework 312 may include a plurality of covering strips 320 (shown in FIG. 8) positioned along the first end region 311 of the tubular framework 312 with the one or more tie strings 340 interposed between adjacent ones of the plurality of covering strips 320. While it is shown that the plurality of covering strips 320 are positioned along the first end region 311, it may be contemplated that the plurality of covering strips 320 may be positioned along at least one of the first end region 311, the second end region 313, or the medial region 314. The plurality of covering strips 320 may be integral portions of the polymeric covering 316, or the covering strips 320 may be separate from the polymeric covering 316 that covers the tubular framework 312.

In some cases, each of the plurality of covering strips 320 may extend longitudinally along the first end region 311 of the tubular framework 312. In some cases, the plurality of covering strips 320 may fully cover the first end region 311 of the tubular framework 312 in the radially collapsed configuration 330, shown in FIG. 7. In some cases, the plurality of covering strips 320 may be separate from one another when the tie strings 340 are removed to permit the tubular framework 312 (e.g., the first end region 311 of the tubular framework 312) to radially expand to the radially expanded configuration 332, as shown in FIG. 8. The plurality of covering strips 320 may each include a first longitudinal edge 321 and a second longitudinal edge 323 with exposed portions 322 of the tubular framework 312 located therebetween, in the radially expanded configuration. The tie strings 340 may be attached with the plurality of covering strips 320 in the radially collapsed configuration 330 or otherwise retain the plurality of covering strips 320 joined together in the radially collapsed configuration 330. In some cases, the one or more tie strings 340 may include a tie string interposed between adjacent ones of the plurality of covering strips 320. In some cases, when the tubular framework 312 is in the radially collapsed configuration 330, the second longitudinal edge 323 may be adjacent the first longitudinal edges 321 of an adjacent covering strip 320 to fully cover the first end region 311 of the tubular framework 312. In some cases, the second longitudinal edge 323 of each of the plurality of covering strips 320 may be overlapped with the first longitudinal edge 321 of an adjacent one of the plurality of covering strips 320, thereby providing a full covering over the first end region 311 when the tubular framework 312 is in the radially collapsed configuration 330. Thus, when the tubular framework 312 is in the radially collapsed configuration 330, tissue may be precluded from growing into the interstices 319 of the first end region 311 where the plurality of covering strips 320 are located.

In some cases, the stent 300 may be placed across a stricture in a body vessel. When desired to expose portions of the tubular framework 312 for tissue ingrowth therein, the retrieval tether 345 may be pulled like a draw string, which may pull on the one or more tie strings 340. The one or more tie strings 340 may be embedded within the polymeric covering 316, such as the plurality of covering strips 320, and thus when the retrieval tether 345 is pulled, the one or more tie strings 340 may be removed from the polymeric covering 316, thereby permitting portions of the polymeric covering 316 (e.g., the plurality of covering strips 320) to separate from one another to expose portions of the first end region 311 of the tubular framework 312 therebetween. The exposed portions 322 may then permit tissue ingrowth therein to provide anti-migration capabilities to the stent 300.

The exposed portions 322 (e.g., bare or uncovered) of the tubular framework 312 may be free from or devoid of the polymeric covering 316, and may expose the interstices 319 of the first end region 311 of the tubular framework 312. Tissue may be permitted to grow into the interstices 319 in the first end region 311, thereby reducing migration of the stent 300.

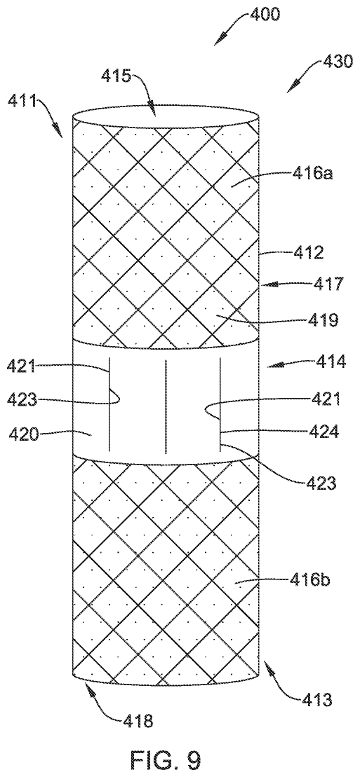

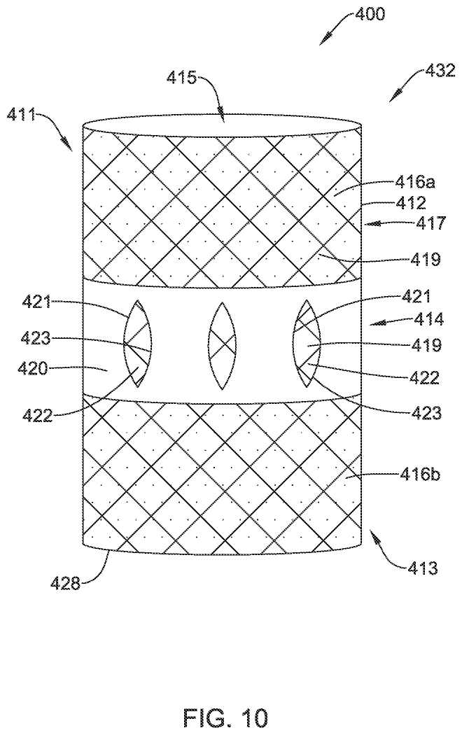

FIG. 9 is a side view of an illustrative stent 400 in a radially collapsed configuration 430, and FIG. 10 is a side view of the illustrative stent 400 of FIG. 9 in a radially expanded configuration 432. In some cases, the stent 400 may be formed from a radially expandable tubular framework 412 (generally referred to herein as tubular framework 412), having a radially outward surface 417 and a radially inward surface 418. While the stent 400 is described as generally tubular, it is contemplated that the stent 400 may take any cross-sectional shape desired. The tubular framework 412 may further include a first end region 411, a second end region 413, and a medial region 414 positioned between the first end region 411 and the second end region 413. The tubular framework 412 may include a lumen 415 extending therethrough from the first end region 411 to the second end region 413.

It is contemplated that the tubular framework 412 can be made from a number of different materials such as, but not limited to, metals, metal alloys, shape memory alloys and/or polymers, as desired, enabling the tubular framework 412 to be expanded into shape when accurately positioned within the body. In some instances, the material may be selected to enable the tubular framework 412 to be removed with relative ease as well. For example, the tubular framework 412 can be formed from alloys such as, but not limited to, Nitinol and Elgiloy®. Depending on the material selected for construction, the tubular framework 412 may be self-expanding (i.e., configured to automatically radially expand when unconstrained). In some embodiments, filaments or wires may be used to make the tubular framework 412, which in some cases may be composite filaments or wire, for example, having an outer shell made of Nitinol having a platinum core. It is further contemplated the tubular framework 412 may be formed from polymers including, but not limited to, polyethylene terephthalate (PET). In some embodiments, the tubular framework 412 may be self-expanding while in other embodiments, the tubular framework 412 may be expanded by an expansion device (such as, but not limited to a balloon inserted within the lumen 415 of the tubular framework 412). As used herein the term “self-expanding” refers to the tendency of the stent to return to a preset diameter when unrestrained from an external biasing force (for example, but not limited to, a delivery catheter or sheath). In some instances, the tubular framework 412 may include a one-way valve, such as an elastomeric slit valve or duck bill valve, positioned within the lumen 415 thereof to prevent retrograde flow of gastrointestinal fluids.

The tubular framework 412 may be configured to expand from a radially collapsed configuration 430, shown in FIG. 9, to a radially expanded configuration 432, shown in FIG. 10. In some cases, the tubular framework 412 may be initially deployed in a body lumen to a partially expanded configuration between the radially collapsed configuration 430 and the radially expanded configuration 432. The stent 400 may be structured to extend across a stricture and to apply a radially outward pressure to the stricture in a body lumen to open the lumen and allow for the passage of foods, fluids, air, etc. When the tubular framework 412 is in the radially collapsed configuration 430, an outer diameter is reduced relative to a partially or fully radially expanded configuration. In some cases, to reduce the diameter of the tubular framework 412 for delivery to the target location, the tubular framework 412 is elongated. For example, when the tubular framework 412 is in the radially collapsed configuration 430, the tubular framework 412 may include a first length and the first outer diameter. When the tubular framework 412 is deployed (moved from a delivery or radially collapsed configuration to a radially expanded configuration), the length of the tubular framework 412 decreases and the outer diameter increases. In some cases, the tubular framework 412 may experience a range of about 20% to about 40% foreshortening (e.g., the percentage by which the length of a stent decreases from its delivery configuration to its radially expanded configuration). It is contemplated that the change in length and/or the change in diameter of the tubular framework 412 may be at least partially dependent on a size (e.g., diameter) of the tubular framework 412. In some cases, a biliary stent may have a deployed diameter in the range of about 8 millimeters to about 10 millimeters, and a collapsed diameter of about 2.5 millimeters to about 3 millimeters. This may correspond to about a 60% to about 80% reduction in diameter from the radially expanded configuration 432 to the radially collapsed configuration 430. This is just an example. The reduction in diameter may be less than 60% or greater than 80%, as desired. In another example, an endoscopy stent may have a radially expanded diameter (e.g., deployed diameter) in the range of about 18 millimeters to about 23 millimeters and a radially collapsed diameter (e.g., delivery diameter) of about 6 millimeters to about 6.5 millimeters. This may correspond to about a 60% to about 80% reduction in diameter from the radially expanded configuration 432 to the radially collapsed configuration 430. This is just an example. The reduction in diameter may be less than 60% or greater than 80%, as desired.

In some cases, the tubular framework 412 may have an interwoven filament structure, fabricated from one or more interwoven filaments defining interstices 419 therebetween. In some cases, the tubular framework 412 may include a single filament knitted or otherwise interwoven with itself to form the tubular framework 412, while in other cases, the tubular framework 412 may include two or more filaments (e.g., a plurality of filaments) interwoven (e.g., wound, braided, looped, etc.) together to form the tubular framework 412. In some cases, the radially inward surface 418 and/or the radially outward surface 417 of the tubular framework 412 may be provided by the filament(s). In some cases, the radially inward surface 418 and/or the radially outward surface 417 may be entirely, substantially, or partially covered with a polymeric covering, such as a first polymeric covering 416a and/or a second polymeric covering 416b, or any other suitable type of covering or coating. The first polymeric covering 416a and the second polymeric covering 416b may be generally referred to herein as the polymeric covering 416. The polymeric covering 416 may be configured to help reduce food impaction and/or tumor or tissue ingrowth into the lumen 415 of the stent 400. In some cases, the polymeric covering 416 may be formed from any suitable material. For example, the polymeric covering 416 may be formed from silicone, polytetrafluoroethylene, polyurethane, or the like, or other materials including those disclosed herein. In some cases, the first end region 411 and the second end region 413 may include the polymeric covering 416 fully covering the interstices 419 within the first end region 411 and the second end region 413. For example, the first polymeric covering 416a may extend throughout the entire first end region 411 (both longitudinally and circumferentially) and span across the interstices in the first end region 411, and the second polymeric covering 416b may extend throughout the entire second end region 413 (both longitudinally and circumferentially) and span across the interstices in the second end region 413.

In some cases, the tubular framework 412 may include a covering strip 420 positioned along the medial region 414 of the tubular framework 412. While it is shown that the covering strip 420 is positioned along the medial region 414, it may be contemplated that the covering strip 420 may be positioned along at least one of the first end region 411, the second end region 413, and/or the medial region 414. The covering strip 420 may be separate from the polymeric covering 416 that covers the first end region 411 and the second end region 413. In some cases, the covering strip 420 may be coupled to the polymeric covering 416. In some cases, the covering strip 420 and the polymeric covering 416 may be one monolithic structure. The covering strip 420 may be configured to help reduce food impaction and/or tumor or tissue ingrowth into the lumen 415 of the stent 100. In some cases, the covering strip 420 may be formed from any suitable material. For example, the covering strip 420 may be formed from silicone, polytetrafluoroethylene, polyurethane, or the like. In some cases, coupling the covering strip 420 to the tubular framework 412 may include adhesive bonding, thermal bonding, molding, coating, dip coating, extruding, or the like. In some cases, the covering strip 420 is positioned over the tubular framework 412, similar to a sleeve. These are just examples.

In some cases, the covering strip 420 may extend circumferentially around the tubular framework 412, and extend from the first polymeric covering 416a to the second polymeric covering 416b along the medial region 414 of the tubular framework 412. As stated herein, in some cases the covering strip 420 may be the portion of the polymeric covering 416 covering the medial region 414 of the tubular framework 412. The covering strip 420 may fully cover the medial region 414 of the tubular framework 412 in the radially collapsed configuration 430, as shown in FIG. 9. The covering strip 420 may include a plurality of slits 424 within the covering strip 420. When the tubular framework 412 is in the radially collapsed configuration 430, the plurality of slits 424 may remain closed, thereby keeping the medial region 414 of the tubular framework 412 fully covered. Thus, when the tubular framework 412 is in the radially collapsed configuration 430, tissue may be precluded from growing into the interstices 419 of the medial region 414 of the tubular framework 412. Thus, a first edge 421 of a slit 424 may abut or be juxtaposed with a second edge 423 of a slit when the tubular framework 412 is in the radially collapsed configuration 430.

As shown in FIG. 10, when the tubular framework 412 is deployed (moved from a delivery or radially collapsed configuration to a radially expanded configuration), the length of the tubular framework 412 may decrease and the outer diameter may increase. In some cases, the tubular framework 412 may be placed across a stricture in a body vessel. The tubular framework 412 may move from the radially collapsed configuration 430 to the radially expanded configuration 432 as the stricture resolves, such that a diameter of the lumen through the stricture increases. In such cases, the medial region 414 of the tubular framework 412 may further expand from an initially deployed diameter to a further radially expanded diameter over a period of time (e.g., several days and/or weeks post implantation).

When the tubular framework 412 expands to the radially expanded configuration 432, the plurality of slits 424 within the covering strip 420 may be configured to separate from one another to expose portions 422 of the tubular framework 412 therebetween. In other words, the first edge 421 of a slit 424 may move away from the second edge 423 of the slit 424 to exposed portions of the tubular framework 412 therebetween. The exposed portions 422 (e.g., bare or uncovered) of the tubular framework 412 may be free from or devoid of the polymeric covering 416, and may expose the interstices 4419 of the medial region 14 of the tubular framework 412. Tissue may be permitted to grow into the interstices 419 in the medial region 414 when the tubular framework 412 is in the radially expanded configuration 432, thereby reducing migration of the stent 400.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic Nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent comprising:
a radially expandable tubular framework having a radially outward surface, a radially inward surface, a first end region, a second end region, a medial region positioned between the first end region and the second end region, and a lumen extending therethrough;
wherein the radially expandable tubular framework is configured to expand from a radially collapsed configuration to a radially expanded configuration; and
a plurality of covering strips positioned along at least one of the first end region, the medial region, and the second end region, the plurality of covering strips fully covering the radially expandable tubular framework in the radially collapsed configuration, and the plurality of covering strips configured to separate from one another in the radially expanded configuration to expose portions of the radially expandable tubular framework therebetween.

2. The stent of claim 1, wherein each of the plurality of covering strips extends longitudinally along the medial region of the stent, the plurality of covering strips fully covering the medial region in the radially collapsed configuration, and the plurality of covering strips configured to separate from one another in the radially expanded configuration to expose portions of the medial region of the radially expandable tubular framework therebetween.

3. The stent of claim 2, wherein a first longitudinal edge of each of the plurality of covering strips is secured to the expandable tubular framework and a second longitudinal edge of each of the plurality of covering strips is unsecured to the expandable tubular framework.

4. The stent of claim 3, wherein the second longitudinal edge of each of the plurality of covering strips is overlapped with the first longitudinal edge of an adjacent one of the plurality of covering strips in the radially collapsed configuration.

5. The stent of claim 4, wherein the second longitudinal edges are located radially outward of the first longitudinal edges in the radially collapsed configuration.

6. The stent of claim 1, wherein each of the plurality of covering strips extends circumferentially around the medial region of the stent, the plurality of covering strips fully covering the medial region in the radially collapsed configuration, and the plurality of covering strips configured to separate from one another in the radially expanded configuration to expose portions of the medial region of the radially expandable tubular framework therebetween.

7. The stent of claim 6, wherein a first circumferential edge of each of the plurality of covering strips is secured to the expandable tubular framework and a second circumferential edge of each of the plurality of covering strips is unsecured to the expandable tubular framework.

8. The stent of claim 7, wherein the second circumferential edge of each of the plurality of covering strips is overlapped with the first circumferential edge of an adjacent one of the plurality of covering strips in the radially collapsed configuration.

9. The stent of claim 1, wherein the radially expandable tubular framework is formed of one or more interwoven filaments defining interstices therebetween, wherein tissue is permitted to grow into the interstices in the medial region in the radially expanded configuration and tissue is precluded from growing into the interstices in the medial region in the radially collapsed configuration.

10. The stent of claim 9, wherein the first end region includes a polymeric cover fully covering the interstices in the first end region, and the second end region includes a polymeric cover fully covering the interstices in the second end region.

11. The stent of claim 10, wherein the plurality of covering strips are separate from the polymeric cover of the first end region and the polymeric cover of the second end region.

12. The stent of claim 1, wherein the plurality of covering strips are positioned along the first end region, the plurality of covering strips fully covering the first end region in the radially collapsed configuration, and the plurality of covering strips configured to separate from one another in the radially expanded configuration to expose portions of the first end region of the radially expandable tubular framework therebetween.

13. The stent of claim 12, further comprising one or more tie strings attached with the plurality of covering strips in the radially collapsed configuration, wherein the one or more tie strings are removable from the plurality of covering strips to permit the first end region to radially expand to the radially expanded configuration.

14. The stent of claim 13, wherein the one or more tie strings includes a tie string longitudinally interposed between adjacent ones of the plurality of covering strips.

15. A stent comprising:
a radially expandable tubular framework having a radially outward surface, a radially inward surface, a first end region, a second end region, a medial region positioned between the first end region and the second end region, and a lumen extending therethrough;
wherein the radially expandable tubular framework is configured to expand from a radially collapsed configuration to a radially expanded configuration;
a first polymeric covering fully covering the first end region;
a second polymeric covering fully covering the second end region; and
a plurality of covering strips positioned along the medial region, the plurality of covering strips fully covering the medial region of the radially expandable tubular framework between the first polymeric covering and the second polymeric covering in the radially collapsed configuration, and the plurality of covering strips configured to separate from one another in the radially expanded configuration to expose portions of the medial region of the radially expandable tubular framework therebetween.

16. The stent of claim 15, wherein the plurality of covering strips extend longitudinally from the first polymeric covering to the second polymeric covering.

17. The stent of claim 15, wherein each of the plurality of covering strips is overlapped with an adjacent one of the plurality of covering strips in the radially collapsed configuration.

18. A method of using a stent, comprising:
implanting a stent across a stricture in a body lumen, the stent including:
a radially expandable tubular framework having a radially outward surface, a radially inward surface, a first end region, a second end region, a medial region positioned between the first end region and the second end region, and a lumen extending therethrough;
a plurality of covering strips positioned along at least one of the first end region, the medial region, and the second end region;
permitting the stent to initially expand to a first radially expanded configuration within the body lumen upon initial implantation across the stricture, wherein the plurality of covering strips fully cover the radially expandable tubular framework in the first radially expanded configuration; and
permitting the stent to further radially expand from the first radially expanded configuration to a second radially expanded configuration within the body lumen over a period of time, wherein the plurality of covering strips are separated from one another in the second radially expanded configuration to expose portions of the radially expandable tubular framework therebetween.

19. The method of claim 18, wherein the plurality of covering strips are overlapped with one another in the first radially expanded configuration, and the plurality of covering strips are spaced apart from one another in the second radially expanded configuration.

20. The method of claim 18, further comprising removing a tie string between adjacent one of the plurality of covering strips to permit the radially expandable tubular framework to radially expand from the first radially expanded configuration to the second radially expanded configuration.
